(19) United States
(12) Patent Application Publication
(10) Pub. No.: US 2008/0070292 A1
(43) Pub. Date: Mar. 20, 2008

(54) Sieve for Bio-Impactor, Bio-Impactor Equipped with Such a Sieve

(75) Inventors: Pierre Bridenne, Forges les Bains (FR); Pascale Coffinier, Lardy (FR)

Correspondence Address: YOUNG & THOMPSON, 745 SOUTH 23RD STREET, 2ND FLOOR, ARLINGTON, VA, 22202, US

(73) Assignee: ACANTHE, LARDY (FR)

(21) Appl. No.: 11/794,548

(22) PCT Filed: Dec. 21, 2005
(86) PCT No.: PCT/FR05/03218

§ 371 Date: Jul. 2, 2007

(30) Foreign Application Priority Data
Dec. 31, 2004 (FR) 0414110

(51) Int. Cl. C12M 1/04

(52) U.S. Cl. 435/287.3

(57) ABSTRACT

The invention concerns a sieve (2) for bio-impactor (1), characterized in that it comprises air passages (6) from upstream (M) towards downstream (V) of the sieve (2), the passages (6) having a gradually tapering cross-section from an inlet (11) towards an outlet (12). Preferably, the inlets (11) are mutually connected forming a sharp edge (13) over their entire periphery, and the outlets (12) are circular. In a preferred embodiment, the inlets (11) are hexagonal, and the passages (6) have a cylindrical end (16) proximate the outlet (12) surrounded by a thin material thickness. Such a sieve is particular adapted to receive large particles, such as moulds and yeasts in a culture medium (4) supported by the bio-impactor (1).

This invention relates to an improved sieve for a bio-impactor.

Bio-impactors are suitable for removing microorganisms from the air in order to subject them to microbiological analysis. The air is entrained in the direction of a culture medium on which the microorganisms that it contains are deposited by inertia. In order to lend sufficient speed to the microorganisms and therefore sufficient inertia for them to be deposited on the culture medium, a sieve arranged upstream of the culture medium is used. The sieve is composed of a plate perforated by cylindrical air passages. As a result, the passages have a smaller section than the front surface of the sieve, and the air is accelerated.

The lightest microorganisms, such as bacteria and viruses, easily pass through the sieve following the current of accelerated air. However, some of the heaviest microorganisms, such as moulds and yeasts, do not pass through the sieve. Because of their greater inertia, they are not carried along by the flow of accelerated air and are deposited on the front surface of the sieve. It is estimated that approximately fifty percent of the microorganisms measuring more than a micron do not pass through the sieve. Microbiological analyses carried out on samples taken with such sieves may be distorted.

The purpose of the invention is to propose an acceleration device which allows almost all of the microorganisms in the air, or at least a greater number than with existing sieves, to be sampled with a view to carrying out more precise analyses.

According to the invention, such a device is a sieve for a bio-impactor, characterized in that it comprises air passages from upstream to downstream of the sieve, the passages having a section that is progressively narrowed from an inlet opening towards an outlet opening.

The inlet opening and the outlet opening can have a same geometric shape, which can make their design and/or production easier. Polygonal inlet openings are preferably formed such that they can easily be joined together. Thus, the sieve no longer has a front surface on which the microorganisms can be deposited, but only ridges forming the sides of the inlet openings. The ridges are preferably sharp. Inlet openings in the shape of a triangle, square or hexagon are suitable for forming such a sieve. However, a residual front surface can remain at the periphery of the sieve, in particular if the sieve is round and if the openings are polygonal.

In order that each quantity of air sampled respectively in each of the passages is approximately identical for all the passages, it is preferable to provide passages that are all approximately identical in shape and size. In particular, it is preferable to provide inlet openings that are approximately identical in shape and in size.

Also preferably, it can be envisaged that the outlet opening is circular. In fact, in this zone where the air is accelerated, the presence of angles on the walls of the passage can slow down the displacement of air, create disturbances in its flow, and/or form zones where certain microorganisms would be deposited.

It is also preferred that the passage comprises a cylindrical zone adjacent to the outlet opening. In this cylinder, the direction and value of the air speeds can stabilize. Approximately unidirectional air flows can thus be obtained under certain flow rate conditions.

The sieve can moreover form a space around the passages, this space being open downstream. The air which strikes the culture medium is substantially pushed back upstream in the direction of the sieve. Greater disturbances of this air flow can detach the microorganisms from the culture medium, and carry them along beyond the culture medium. The analyses can thus be distorted. Thanks to this space, the air which strikes the culture medium and which is substantially pushed back upstream in the direction of the sieve can escape by circulating not only in the gap between sieve and culture medium, but also in the complementary space thus formed between the air passages. The flow of this air is thereby facilitated and it is less disturbed.

Other features and advantages of the invention will also become apparent from the description below which relate to non-limitative examples.

In the attached drawings:

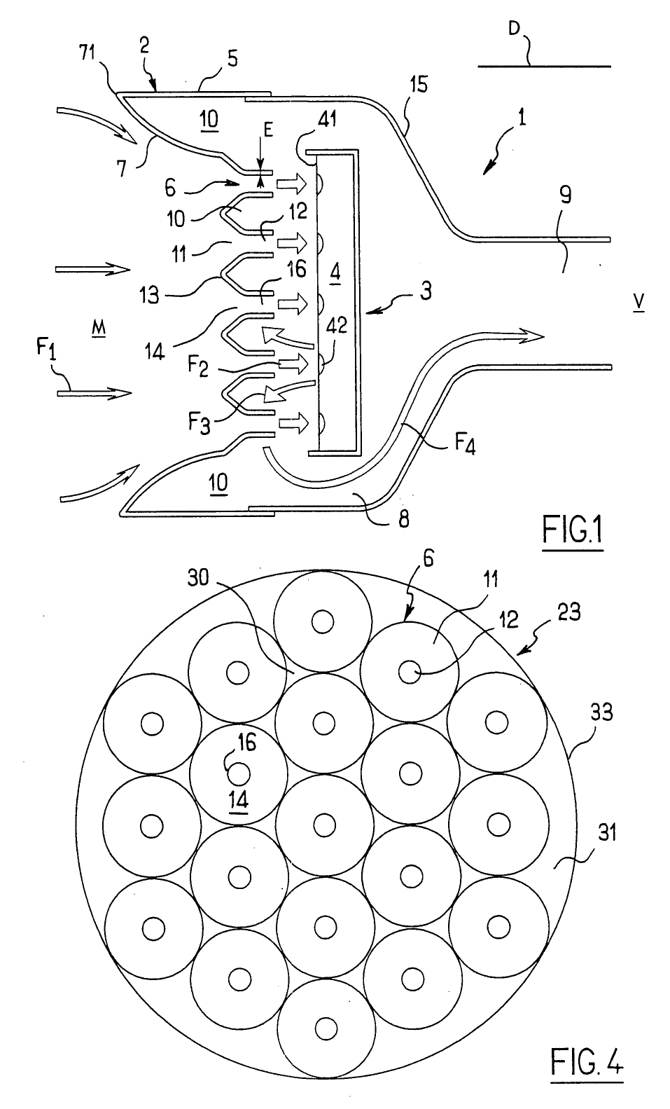

FIG. 1 is a sectional diagrammatic view of a bio-impactor equipped with a sieve according to the invention;

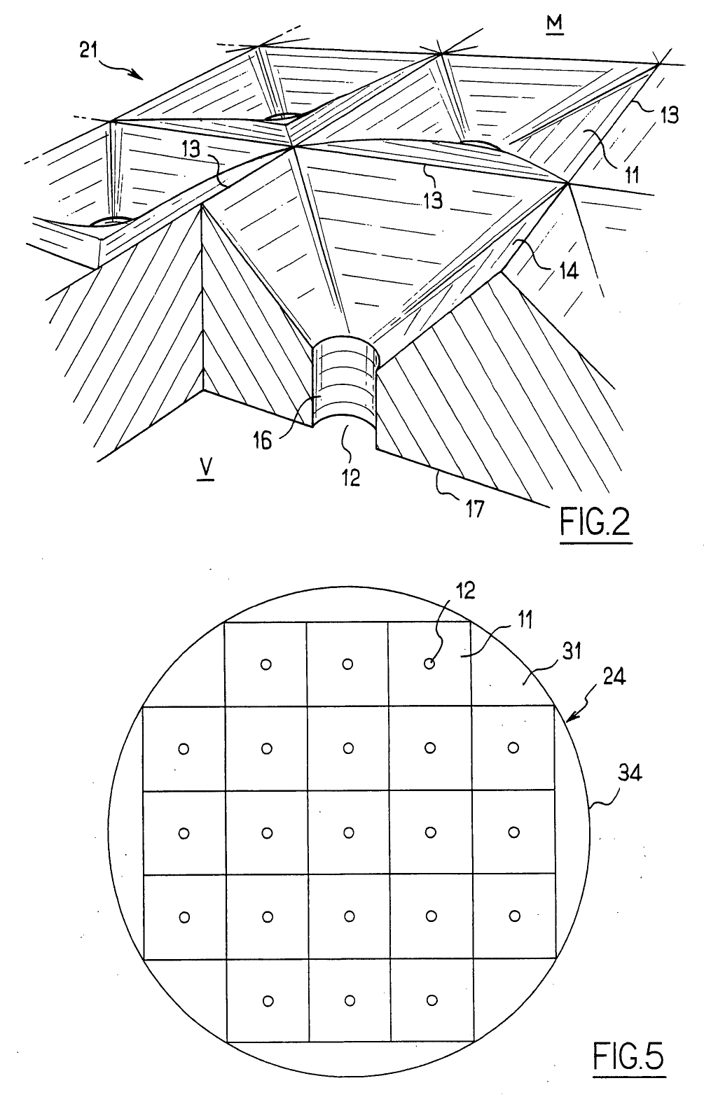

FIG. 2 is a diagrammatic, partly enlarged, perspective and sectional view of a sieve according to the invention;

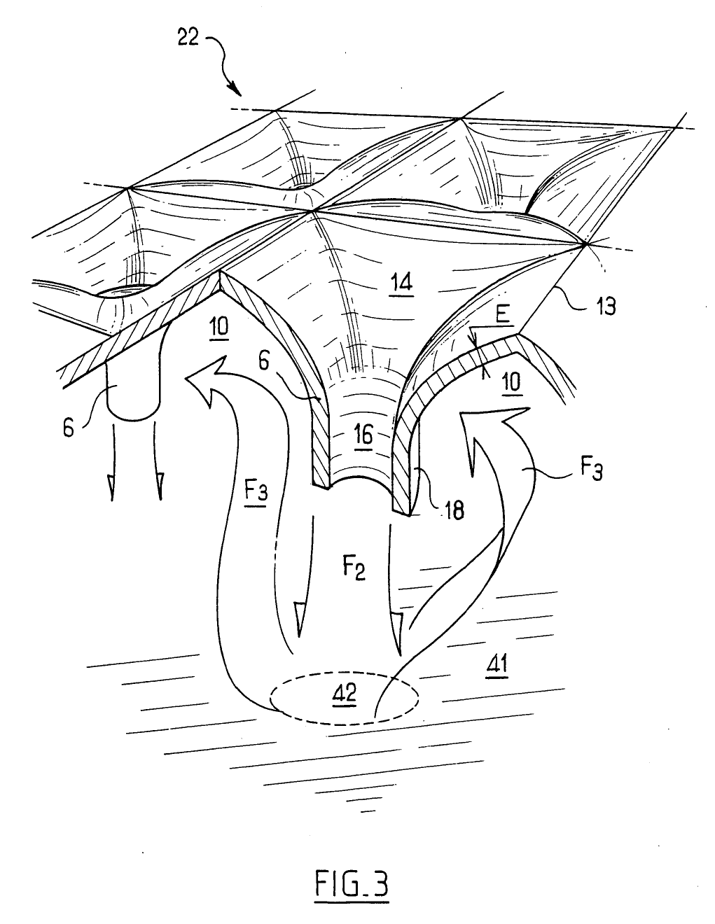

FIG. 3 is a view similar to that in FIG. 2 of a sieve according to the invention, forming a space around the air passages;

FIG. 4 is a plan view of a circular sieve according to the invention, comprising circular inlet and outlet openings;

FIG. 5 is a plan view of a circular sieve according to the invention, comprising square inlet openings and circular outlet openings;

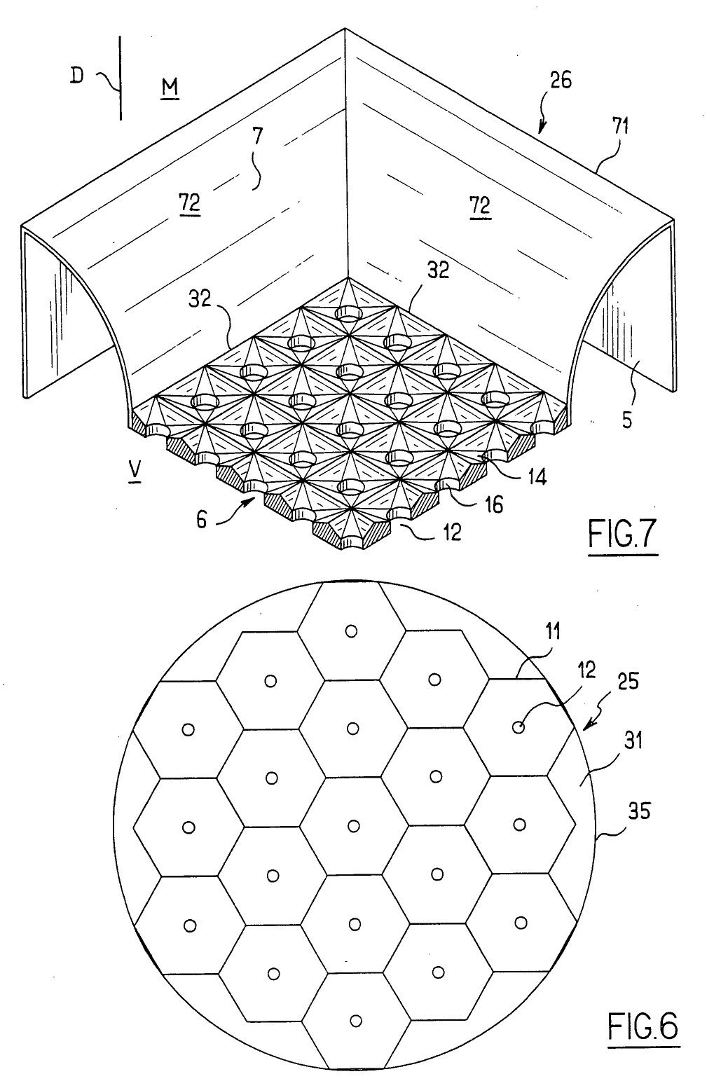

FIG. 6 is a plan view of a circular sieve according to the invention, comprising hexagonal inlet openings and circular outlet openings;

FIG. 7 is a perspective and sectional view of a square sieve according to the invention, comprising square inlet openings, circular outlet openings and a cowl with convex surfaces;

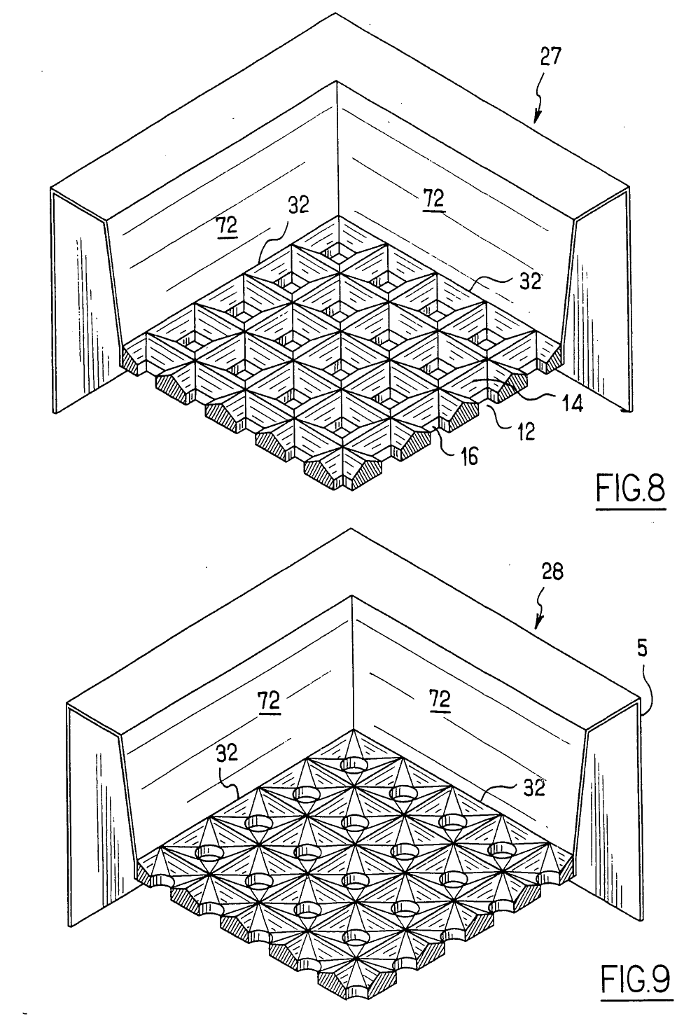

FIG. 8 is a perspective and sectional view of a square sieve according to the invention, comprising square inlet openings, square outlet openings and a cowl with flat surfaces; and,

FIG. 9 is a perspective and sectional view of a square sieve according to the invention, comprising square inlet openings, circular outlet openings and a cowl with flat surfaces.

FIG. 1 represents very diagrammatically a bio-impactor 1 equipped with a sieve 2 according to the invention. The bio-impactor extends in a longitudinal direction D, between upstream M and downstream V. The bio-impactor has a Petri dish 3 filled with a culture medium 4. The culture medium is arranged opposite the sieve 2. The sieve comprises passages 6 for the air aspirated by the bio-impactor and a peripheral cowl 7 arranged upstream M of the passages. A skirt 5 extends downstream from a peripheral upstream edge 71 of the cowl 7. The skirt allows the sieve 2 to be mounted by substantially airtight fitting onto a body 15 of the bio-impactor. For purposes of illustration, only a small number of relatively wide passages is shown. In practice, there are typically several hundred passages.

The passages 6 are progressively narrowed from upstream M to downstream V. The cowl 7 itself is also progressively narrowed from upstream M to downstream V. The passages 6 are uniformly distributed opposite a culture surface 41 of the culture medium 4. The outlet openings are all at the same longitudinal distance from the culture surface. The culture surface 41 is perpendicular to the longitudinal direction D. Each passage comprises an inlet opening 11 and an outlet opening 12 for the air in the passage. Each inlet opening joins onto the inlet opening or openings of adjacent passages. The sides of the inlet openings form between them sharp ridges 13 which are perpendicular to the longitudinal direction D. The cowl 7 allows air to be sampled over a cross-section greater than that of the culture surface and this air to be taken towards the passages 6.

Each passage 6 comprises a progressively narrowed upstream zone 14, adjacent to the inlet opening 11, and a cylindrical downstream zone 16, adjacent to the outlet opening 12. The upstream zone 14 and the downstream zone 16 are continuous and adjacent to each other,. i.e. the downstream end of the upstream zone 14 forms a connecting circle the diameter of which is identical to that of the cylinder formed by the downstream zone 16, this connecting circle also being the upstream end of the downstream zone. The downstream zone 16 is approximately centred with the upstream zone 14. The generatrices of the cylinder are approximately perpendicular to the ridges 13 and to the surface 41 of the culture medium 4. The generatrices of the cylinder are moreover parallel to the longitudinal direction D.

The air to be analyzed is firstly aspirated in the direction of arrows F1, between the peripheral walls of the cowl 7 and passes through the passages 6. On leaving each passage, the air forms a flow, shown by arrows F2, which strikes the surface 41 of the culture medium 4, in an impact zone 42 opposite each outlet opening 12. At least some of the microorganisms in the air are deposited by inertia in this impact zone. The air bounces off the surface 41 substantially in the direction of the sieve according to arrows F3 (partially represented), then it is at least partially extracted via a circulation zone surrounding the air passages at the periphery 8 of the Petri dish 3 in the direction shown by arrow F4.

FIG. 2 shows a detail of a first embodiment for a sieve 21 according to the invention. We will describe it only insofar as it differs from or supplements the description of the previously-described sieve 2.

The sieve 21 of FIG. 2 comprises square inlet openings 11 with identical dimensions and arranged in a chequered fashion. These openings are joined and form sharp ridges 13 between them. The surfaces of the upstream zones 14 are regular surfaces between the ridges 13 and the cylinder of the downstream zone 16. The ridges are coplanar and parallel to a downstream plane surface 17 of the sieve. The downstream surface 17 is parallel to the surface 41 of the culture medium 4. The outlet openings 12 of the passages 6 are formed in the downstream surface 17.

FIG. 3 shows a detail of a second embodiment for a sieve 22 according to the invention. We will describe it only insofar as it differs from or supplements the description of the previously-described sieve 21.

The sieve 22 of FIG. 3, like the sieve 21 of FIG. 2, comprises square inlet openings 11 forming sharp ridges between them. The surfaces of the upstream zones 14 are surfaces with a convex profile extending between the ridges 13 and the cylinder of the downstream zone 16, such that on the connecting circle the generatrices of the cylinder 16 are tangential to the convex profile of the upstream zone 14. The disturbances which may be created in the air flow by passing over a ridge between the upstream zone 14 and the downstream zone 16 are thus avoided.

At all points, the sieve has an approximately constant material thickness E which is small relative to the dimensions of a passage. The small thickness E creates favourable hydrodynamic conditions around the outlet of the passages. Moreover, the sieve thus forms a space 10 around the passages.

The air carried along as shown by F2 towards the surface 41 of the culture medium 4 deposits the microorganisms in the impact zone 42. The air is then pushed back substantially downstream as shown by F3, around an air flow F2 which follows it and pushes it. The space 10 then allows the air to circulate under the sieve and around the passages. While still positioning the outlet openings 12 sufficiently close to the surface 41 of the culture medium, a volume is thus obtained which allows the pushed back air to circulate while still suppressing or at least by reducing the disturbances which would arise in the absence of the complementary circulation space 10.

The Petri dishes generally used are circular in shape. As the outlet openings must be approximately uniformly distributed opposite the Petri dish, in this case, a sieve should be provided with an approximately circular shape. Embodiments for circular sieves according to the invention will therefore now be described with reference to FIGS. 4 to 6.

FIG. 4 shows a sieve 23 comprising nineteen passages 6 the inlet opening of which is circular. The passages are defined by revolution surfaces 14, 16. The inlet openings 11 are identical in diameter and arranged in staggered rows so that they each touch their neighbours. The upstream zone 14 can be formed by a regular, i.e. tapered, surface or by a surface with a convex profile. The nineteen inlet openings 11 are arranged so as to be inscribed in a circle 33 the diameter of which is approximately identical to the diameter of the Petri dish used.

Such a sieve is easy to design and to produce. However, it suffers from the disadvantage that front surfaces 30 remain between the inlet openings. These surfaces are reduced and such a sieve nevertheless brings about a noticeable improvement compared with the sieves of the prior art.

The use of polygonal openings, although they can be more expensive to design and produce, depending on the chosen embodiment, makes it possible to overcome this disadvantage. Three types of regular polygon can be used in particular, triangles, squares, as shown in FIGS. 2 and 3, or hexagons, in order to form a sieve with inlet openings that are identical in shape and size.

Thus, FIG. 5 shows a sieve 24 the inlet openings of which are 21 joined squares, i.e. they leave no front surface between them. The squares are inscribed in a circle 34 the diameter of which is approximately identical to the diameter of the Petri dish used.

Similarly, FIG. 6 shows a sieve 25 the inlet openings of which are 19 joined hexagons, leaving no front surface between them. The hexagons are inscribed in a circle 35 the diameter of which is approximately identical to the diameter of the Petri dish used.

The circular sieves of FIGS. 4 to 6 allow a peripheral front surface 31 to remain. This front surface 31 can be reduced by increasing the numbers of circles, squares or hexagons. The hexagons have the advantage, compared with the square and for the same number of passages, of reducing to the maximum extent the peripheral front surface 31.

FIGS. 7 to 8 show three embodiments for square sieves. These sieves are particularly suitable for use with square Petri dishes. They will be described insofar as they differ from, or supplement, the descriptions of previously-described embodiments.

These three embodiments each have 25 inlet openings arranged in a chequered square. A wall 72 of a cowl 7 extends from each of the four edges 32 of the chequered squares, widening upstream M. For greater clarity, only two of these walls 72 are shown in the figures.

The sieve 26, shown in FIG. 7, comprises convex walls 72 such that along the edges 32 of the chequered square the walls 72 are tangential to the longitudinal direction D.

The sieves 27 and 28, shown in FIGS. 8 and 9 respectively, have plane, thus trapezoidal, walls 72. However, the cowl is hardly widened with the result that the walls are almost parallel to the longitudinal direction D.

The outlet openings of the sieves 26 and 28 are circular. The upstream surface 14 of each passage 6 is formed by four regular surfaces composed of quarter cones, each respectively between an inlet opening angle and the connecting circle with the cylindrical downstream zone 16, alternated with four triangular plane surfaces, each of the triangular surfaces having a side of the inlet opening for a base.

The outlet openings 12 of the sieve 27 are square. The downstream zone 16 has a constant square section identical to that of the outlet opening. The upstream surface of each opening is composed of four regular surfaces each forming a regular trapezium the bases of which are a side of the inlet opening and an edge of a square section connecting with the downstream zone 16 respectively.

Typically, a passage has an exit diameter of 5/100ths of a mm to 1 mm, with a centre distance of axes of the order of 2 mm, and an axial length of the cylindrical part of 1.5 mm or more generally 3 to 4 times the diameter. The upstream section typically has the same length as the cylindrical part, with a profile slope of 40 to 60° with respect to the axis. The overall diameter of the sieve is typically 50 to 70 mm.

Of course, the invention is not limited to the examples which have just been described and numerous adjustments can be made to these examples without exceeding the scope of the invention.

For example, a sieve can comprise a combination of joined octagonal openings and square openings. It is also possible to use combinations of other types of inlet openings be they regular polygons or not, polygons or not.

1. Sieve (2, 21-28) for a bio-impactor (1), characterized in that it comprises air passages (6) from upstream (M) to downstream (V) of the sieve, the passages having a section that is progressively narrowed from an inlet opening (11) towards an outlet opening (12).

2. Sieve according to claim 1, characterized in that the inlet opening and the outlet opening have the same geometric shape.

3. Sieve according to claim 1, characterized in that the inlet opening has a polygonal shape.

4. Sieve according to claim 3, characterized in that the polygon is regular, the inlet openings being joined.

5. Sieve according to claim 4, characterized in that two joined openings form between them a sharp ridge (13).

6. Sieve according to claim 1, characterized in that the inlet openings are approximately identical in shape and size.

7. Sieve according to claim 1, characterized in that the outlet opening is circular.

8. Sieve according to claim 1, characterized in that the passage has a cylindrical zone (16) adjacent to the outlet opening surrounded by a small thickness (E) of material.

9. Sieve according to claim 1, characterized in that the sieve forms a complementary space (10) for air circulation (F₃) around the passages, this space being open towards the inside.

10. Bio-impactor (1) equipped with a sieve (2, 21-28) according to claim 1.

11. Sieve according to claim 2, characterized in that the inlet opening has a polygonal shape.

12. Sieve according to claim 2, characterized in that the inlet openings are approximately identical in shape and size.

13. Sieve according to claim 3, characterized in that the inlet openings are approximately identical in shape and size.

14. Sieve according to claim 4, characterized in that the inlet openings are approximately identical in shape and size.

15. Sieve according to claim 5, characterized in that the inlet openings are approximately identical in shape and size.

16. Sieve according to claim 2, characterized in that the outlet opening is circular.

17. Sieve according to claim 3, characterized in that the outlet opening is circular.

18. Sieve according to claim 4, characterized in that the outlet opening is circular.

19. Sieve according to claim 5, characterized in that the outlet opening is circular.

20. Sieve according to claim 6, characterized in that the outlet opening is circular.
